(19) United States
(12) Patent Application Publication
(10) Pub. No.: US 2026/0196318 A1
(43) Pub. Date: Jul. 9, 2026

(54) METHOD OF PATIENT REPORTING BASED ON VOICE USING ARTIFICIAL INTELLIGENCE (AI) AND APPARATUS FOR PERFORMING THE METHOD

(71) Applicant: WELT Corp., Ltd, Seoul (KR)

(72) Inventors: Seong Ji KANG, Seoul (KR); Hye Kang ROH, Seoul (KR); Joo Young KIM, Seoul (KR); Do Hyun LEE, Yongin-si (KR); Hwa Young JEONG, lncheon (KR)

(21) Appl. No.: 19/557,655

(22) Filed: Mar. 5, 2026

Related U.S. Application Data
(63) Continuation-in-part of application No. 18/522,432, filed on Nov. 29, 2023, now abandoned.

(30) Foreign Application Priority Data
Aug. 4, 2023 (KR) 10-2023-0102208

(51) Int. Cl. G16H 15/00; G06N 5/02; G10L 15/08; G10L 25/66; G16H 10/60

(52) U.S. Cl. CPC G16H 15/00; G06N 5/027; G10L 15/08; G10L 25/66; G16H 10/60; G10L 2015/088

(57) ABSTRACT

The disclosed embodiments relate to a method and apparatus for patient reporting based on voice using artificial intelligence. The method may include: receiving patient analysis basic information including voice data; inputting the voice data into a voice data analysis AI engine; generating, via a query generating AI engine, a query for additional information from an analysis result of the analysis AI engine; generating report basic information from the basic information, the voice data, the analysis result of the analysis AI engine and the additional information; calculating a reference time-series based on a target accuracy and a disease characteristic; allocating dynamically a portion of memory corresponding to the reference time-series; and generating a patient report based on the report basic information using the patient report basic information and the previous patient report basic information loaded into the allocated memory portion.

### CROSS REFERENCE TO RELATED APPLICATION

This application a continuation-in-part application claiming priority to US non-provisional application No. 18/522,432 filed on November 29, 2023, which is hereby incorporated by reference in its entirety.

### BACKGROUND

### 1. Field of the Invention

The present invention relates to a method of patient reporting based on a voice using artificial intelligence (AI) and an apparatus for performing the method. More particularly, the present invention relates to a method of patient reporting based on a voice using artificial intelligence (AI) for generating patient reports according to various purposes and an apparatus for performing the method.

### 2. Discussion of Related Art

With the development of various smart technologies, data of personal daily activities is recorded, and individual life can be efficiently managed on the basis of the recorded data. In the meantime, health-related data logging is attracting attention due to the increasing interest in healthcare. Many users have already been generating and utilizing various health-related data including data on exercise, diet, sleep, and the like through user devices such as smartphones, wearable devices, and the like. In the past, health-related data was generated and managed only by medical institutions, but now users have begun to generate and manage their own health-related data through user devices such as smartphones and wearable devices.

In many cases, health-related data logging is performed through a wearable device. A wearable device is a user device that is carried by or attached to a user. Due to the development of Internet of things (IoT) and the like, wearable devices are frequently used for collecting health-related data. A wearable device may collect a user’s physical change information and surrounding data of the user through equipment and provide advice required for the user’s healthcare on the basis of the collected data.

A user’s health-related data may include a user biomarker, and research is ongoing on a method of making a medical prescription adaptively to a user on the basis of the user’s health-related data.

As related art, there is Korean Patent No. 10-2425479.

### SUMMARY OF THE INVENTION

An object of the present invention is to solve all of the above problems.

In addition, the present invention is directed to determining a user's disease state based on user voice data and generating a report on a user's state.

In addition, the present invention is also directed to providing patient information for various subjects (guardian, doctor, insurance company) only with a user's voice by generating a patient report based on user voice data.

According to an aspect of the present invention, there is provided a method of patient self-reporting based on a voice using artificial intelligence (AI) comprises receiving, by a patient analysis device, patient analysis basic information, generating, by the patient analysis device, report basic information based on the patient analysis basic information and generating, by a patient report generation device, a patient report based on the report basic information.

Meanwhile, the patient analysis basic information includes voice data, and the patient analysis device inputs the voice data to a voice data analysis AI engine and generates the report basic information.

Further, the patient analysis device further includes a query generating AI engine for acquiring necessary information from a user when the necessary information exists in the voice data analysis AI engine.

According to another aspect of the present invention, there is provided a patient report system for patient self-reporting based on a voice using artificial intelligence (AI), comprises a patient analysis device configured to receive patient analysis basic information and generate report basic information based on the patient analysis basic information and a patient report generating device configured to generate a patient report based on the report basic information.

Meanwhile, the patient analysis basic information includes voice data and the patient analysis device inputs the voice data to a voice data analysis AI engine and generates the report basic information.

Further, the patient analysis device further includes a query generating AI engine for acquiring necessary information from a user when the necessary information exists in the voice data analysis AI engine.

According to another aspect of the present invention, there is provided a non-transitory computer-readable storage medium having computer executable instructions stored thereon, which when executed by one or more processors, cause the one or more processors to perform operations comprising: receiving patient analysis basic information including voice data; inputting the voice data into a voice data analysis AI engine; generating, via a query generating AI engine, at least one query for additional information from an analysis result of the voice data analysis AI engine; generating report basic information from the patient analysis basic information, the voice data, the analysis result of the voice data analysis AI engine and the additional information; calculating, a reference time-series unit based on a target accuracy and the disease characteristic, wherein the reference time-series unit represents a minimum data range for a previous report basic information required to satisfy the target accuracy; allocating dynamically, a portion of memory corresponding to the reference time-series unit; and generating a patient report based on the report basic information using the patient report basic information and the previous patient report basic information loaded into the allocated memory portion.

According to another aspect of the present invention, there is provided a. a patient report system for patient self-reporting based on a voice using artificial intelligence (AI), comprising: a patient analysis device configured to: receive patient analysis basic information; generate, via a query generating AI engine, at least one query for additional information from an analysis result of the voice data analysis AI engine, wherein the patient analysis basic information includes voice data, and the patient analysis device inputs the voice data to a voice data analysis AI engine and generates the report basic information; a patient report generating device configured to: calculate, a reference time-series unit based on a target accuracy and the disease characteristic, wherein the reference time-series unit represents a minimum data range for a previous report basic information required to satisfy the target accuracy; allocate dynamically, a portion of memory corresponding to the reference time-series unit; and generate a patient report based on the report basic information using the patient report basic information and the previous patient report basic information loaded into the allocated memory portion.

Also, in an exemplary embodiment, the system further comprises an information security device. The information security device is implemented to secure information generated from the user device, the patient analysis device and the patient report generating device.

According to another aspect of the present invention, the voice data analysis AI engine comprises: a first engine operating with only the voice data and without considering a medical history record of the user; a second engine operating with considering both the voice data and the medical history record of the user, and work results of both of the first engine and the second engine are utilized for the patient report.

According to exemplary embodiment, the query generating AI engine generates the at least one query based on: at least one part of predetermined information being unable to be filled on the patient report based on the voice data; a reliability value being less than a predetermined range for an answer that generates the patient report with the voice data.

Also, in an exemplary embodiment, the voice data analysis AI engine receives a preprocessed voice data, the preprocessed voice data includes at least one tag of a predetermined disease related to sentences and words of the voice data, and the sentences and words of the voice data are grouped based on the at least one tag for the predetermined disease.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a conceptual diagram illustrating a patient report system according to an embodiment of the present invention.

FIG. 2 is a conceptual diagram illustrating a patient analysis device according to an embodiment of the present invention.

FIG. 3 is a conceptual diagram illustrating a voice data analysis AI engine according to an embodiment of the present invention.

FIG. 4 is a conceptual diagram illustrating a method of generating a report according to an embodiment of the present invention.

FIG. 5 is a conceptual diagram illustrating a method of generating a report according to an embodiment of the present invention.

FIG. 6 is a conceptual diagram illustrating a method of generating patient report information according to an embodiment of the present invention.

FIG. 7 is a conceptual diagram illustrating a method of generating a patient report based on user voice data according to an embodiment of the present invention.

FIG. 8 is a flowchart illustrating a method for patient self-reporting based on a voice using artificial intelligence according to another embodiment of the present invention.

FIG. 9 is a block diagram illustrating that a control signal is transmitted to a pharmaceutical mixer or a supplemental device based on the patient report according to another embodiment of the present invention.

### DETAILED DESCRIPTION OF EXEMPLARY EMBODIMENTS

The detailed description of the present invention will be made with reference to the accompanying drawings showing examples of specific embodiments of the present invention. These embodiments will be described in detail such that the present invention can be performed by those skilled in the art. It should be understood that various embodiments of the present invention are different but are not necessarily mutually exclusive. For example, a specific shape, structure, and characteristic of an embodiment described herein may be implemented in another embodiment without departing from the scope and spirit of the present invention. In addition, it should be understood that a position or arrangement of each component in each disclosed embodiment may be changed without departing from the scope and spirit of the present invention. Accordingly, there is no intent to limit the present invention to the detailed description to be described below. The scope of the present invention is defined by the appended claims and encompasses all equivalents that fall within the scope of the appended claims. Like reference numerals refer to the same or like elements throughout the description of the figures.

Hereinafter, in the present invention, a user is assumed to be a patient for convenience of description, but the user may be interpreted to include not only patients but also non-patients who have not been diagnosed with a specific disease, and these embodiments may also be included in the scope of the present invention.

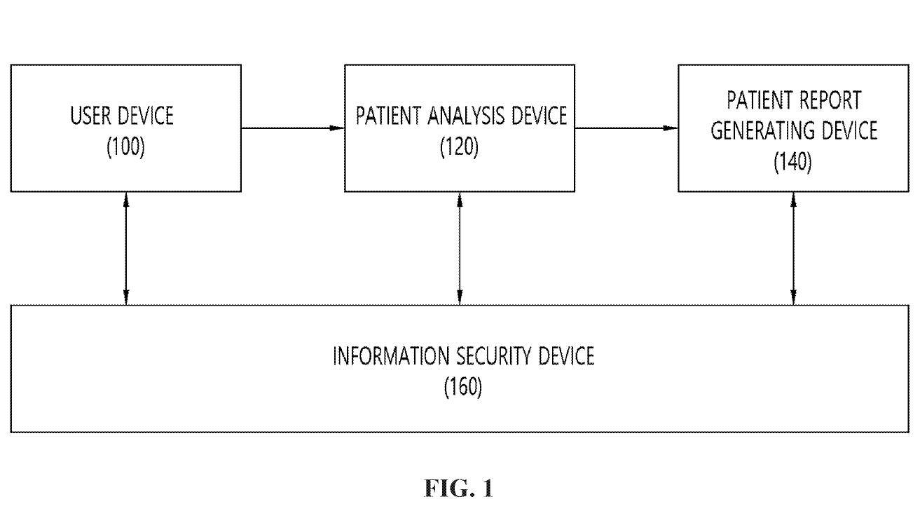

FIG. 1 is a conceptual diagram illustrating a patient report system according to an embodiment of the present invention.

In FIG. 1, a patient report system for generating a patient report based on artificial intelligence (AI) is disclosed.

Referring to FIG. 1, the patient report system may include a user device 100, a patient analysis device 120, a patient report generating device 140, and an information security device 160.

The user device 100 may be a device for transmitting patient analysis basic information, which is based on patient analysis, such as user voice data, to a patient analysis device.

The patient analysis device 120 may be implemented to generate report basic information for generating a patient report based on patient analysis basic information transmitted from a user. For example, the patient analysis device 120 may generate report basic information for generating reports for various subjects (e.g., doctor, guardian, insurance company) based on the user's voice information.

Patient report generating device 140 may be implemented to generate a patient report. The patient report generating device 140 may generate a patient report based on report basic information generated by the patient analysis device. For example, the patient report generating device 140 may generate a first patient report for a doctor, a second patient report for a guardian of the patient, and a third patient report for an insurance company.

The information security device 160 may be implemented to secure information generated from the user device 100, the patient analysis device 120, and the patient report generating device 140.

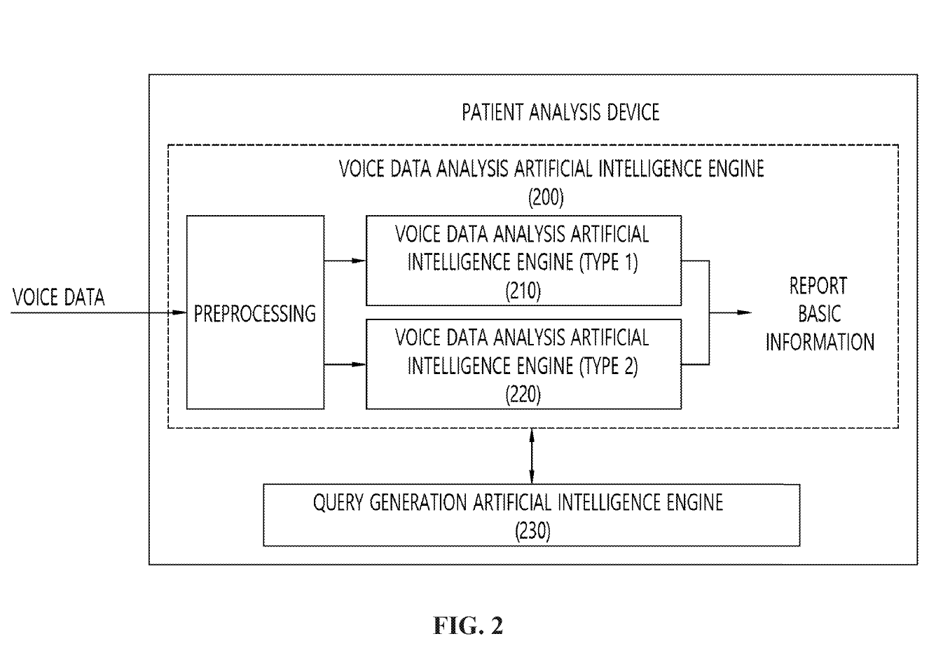

FIG. 2 is a conceptual diagram illustrating a patient analysis device according to an embodiment of the present invention.

In FIG. 2, a patient analysis device implemented to generate report basic information based on patient analysis basic information is disclosed.

Referring to FIG. 2, the patient analysis device may receive voice data as patient analysis basic information. The patient analysis device preprocesses voice data, and the preprocessed voice data may be input to a voice data analysis AI engine 200. The voice data analysis AI engine 200 may generate report basic information based on the preprocessed voice data.

The voice data analysis AI engine 200 may generate different types of report basic information for each report in processing the preprocessed voice data and generating the report basic information. For example, the voice data analysis AI engine 200 may generate first report basic information for a first patient report, second report basic information for a second patient report, and third report basic information for a third patient report.

In addition, the voice data analysis AI engine 200 may be divided into a voice data analysis AI engine (type 1) 210 for preferentially determining a user without a previous medical history record and a voice data analysis AI engine (type 2) for a user with previous medical record 220.

Separate previous medical history data is not input to the voice data analysis AI engine (type 1) 210, and separate previous medical history data is additionally input to the voice data analysis AI engine (type 2) 220, so the report basic information may be generated by additionally considering the previous medical history data.

In addition, according to an embodiment of the present invention, when there is information required for the voice data analysis AI engine 200, a query generating AI engine 230 for acquiring necessary information from a user is additionally included in the patient analysis device.

The query generating AI engine 230 may generate a query in consideration of 1) a part where it is difficult to fill specific information on the report based on the user voice data, 2) a reliability range of an answer that generates a report with the user voice data but has low reliability, and the like.

That is, when generating a report for a specific patient report (first patient report, second patient report, third patient report), in a case in which there is necessary information or the information is acquired but the reliability of the information is low, the query generation engine 230 may generate the query about the corresponding part and transmit the generated query to the user device. The user may generate a response to a query as voice data and transmit the generated response through the user device.

That is, the query generation engine 230 may generate a query in consideration of a required report type, information required for the report, and reliability based on patient voice data, and may provide the generated query to a patient.

The voice data may be input again to the voice data analysis AI engine 200 and converted into the report basic information required for report generation.

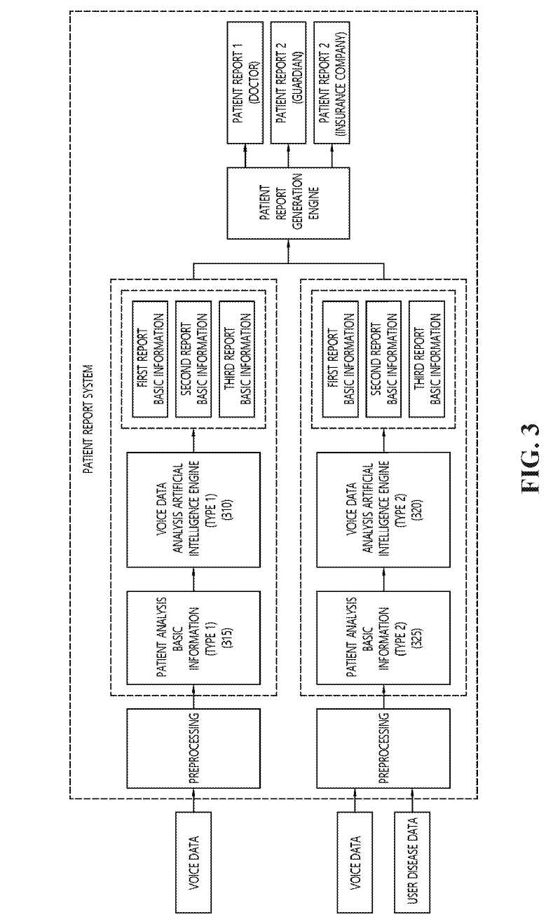

FIG. 3 is a conceptual diagram illustrating a voice data analysis AI engine according to an embodiment of the present invention.

In FIG. 3, operations of a voice data analysis AI engine (type 1) and the voice data analysis AI engine (type 2) are disclosed.

Referring to FIG. 3, detailed operations of a voice data analysis AI engine (type 1) 310 and a voice data analysis AI engine (type 2) 320 are disclosed.

The voice data analysis AI engine (type 1) 310 may perform overall screening for diseases that can be determined overall based on user voice data, and generate report basic information for determining the possibility of various diseases based on the overall screening.

The voice data analysis AI engine (type 2) 320 may generate report basic information for generating a patient report on a disease already possessed by a user based on the user voice data.

In the case of a user who has previously been confirmed to have a disease, the report basic information may be generated through the voice data analysis AI engine (type 1) 310 and the voice data analysis AI engine (type 2) 320.

In the case of a user who has not previously been confirmed to have a disease, the report basic information may be generated through the voice data analysis AI engine (type 1) 310.

That is, a patient report can be generated based on voice data, but a patient report based on the disease already possessed may be generated.

The voice data analysis AI engine (type 1) 310 may receive patient analysis basic information (type 1) 315 in generating the patient analysis information. The patient analysis basic information (type 1) 315 may be generated by preprocessing sentences and words that may be sources for patient report generation, and adding tags of all diseases highly related to sentences and words to sentences and words. In this case, the sentences and words may be grouped based on tags for each of the plurality of diseases. For example, sentences and words related to migraine may be preprocessed in a form including tags for other diseases considering the possibility of other diseases (for example, arteriosclerosis) as well as migraine.

For the report related to migraine, sentences and words related to a tag for migraine may be grouped and grouped into a migraine report group, and the migraine report group may be input to the voice data analysis AI engine (type 1) 310 and used for report generation.

In this way, sentences and words related to each of the plurality of diseases may be grouped based on tags for each of the plurality of diseases and generated as a disease n report group, the disease n report group may be input to the voice data analysis AI engine (type 1) 310, and the voice data analysis AI engine (type 1) 310 may generate report basic information for a target disease requiring a report among a plurality of diseases (disease 1 to disease n).

The voice data analysis AI engine (type 2) 320 may receive patient analysis basic information (type 2) 325 in generating the patient analysis information. The patient analysis basic information (type 2) 325 may include data in which tags for sentences and words related to a target disease are added in order to report the target disease that the user possess. The voice data analysis AI engine (type 2) 320 may generate the report basic information on the target disease based on the patient analysis basic information (type 2) 325 including the tag for the target disease.

For example, first report basic information, second report basic information, and third report basic information may be generated according to the patient report type to be generated. The first report basic information, the second report basic information, and the third report basic information may be input to a patient report generation engine to generate a first patient report, a second patient report, and a third patient report. The first patient report may be a report for a doctor, the second patient report may be a report for a guardian, and the third patient report may be a report for an insurance company.

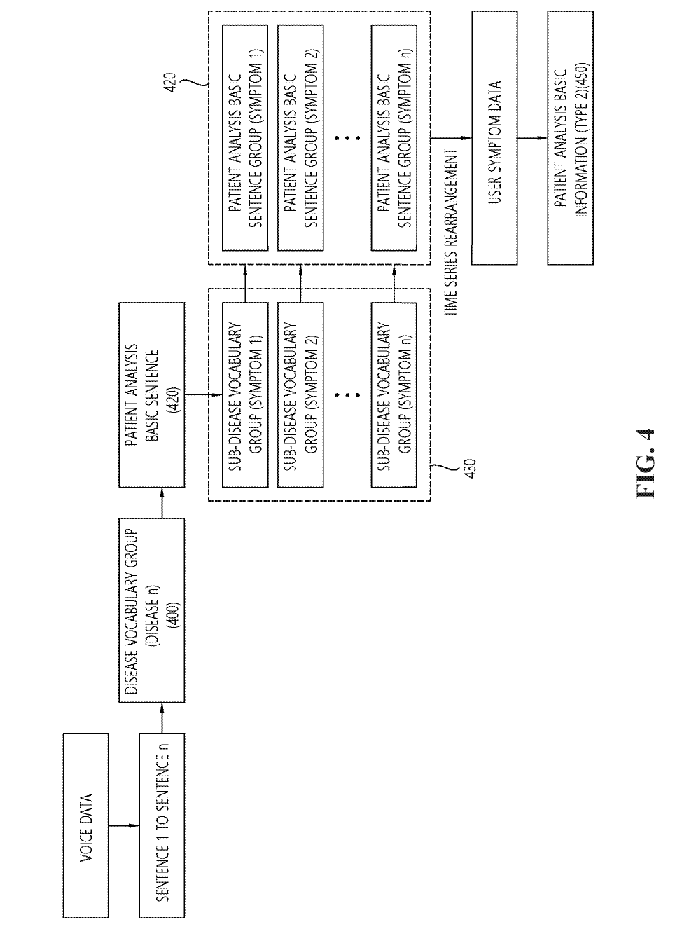

FIG. 4 is a conceptual diagram illustrating a method of generating a report according to an embodiment of the present invention.

In FIG. 4, a method of determining patient analysis basis information for report generation is disclosed. In particular, a method of generating patient analysis basic information (type 2) when there is information on a disease already possessed by a user is disclosed.

Referring to FIG. 4, the user voice data may be textualized. The textualized voice data may be divided into a plurality of sentences. Some of the plurality of sentences may be extracted as patient analysis basic sentences 420 based on a disease vocabulary group (disease n) 400 in consideration of the disease information already possessed by the user. The disease vocabulary group (disease n) 400 may be a group including words highly related to a symptom of a specific disease. That is, a sentence including words corresponding to the disease vocabulary group (disease n) 400 or a sentence highly related to the disease vocabulary group (disease n) 400 among the textualized voice data may be extracted as the patient analysis basic sentences 420.

After the patient analysis basic sentence 420 is extracted, the patient analysis basic sentence 420 may be rearranged through the relationship between the patient analysis basic sentences 420.

The patient analysis basic sentence 420 may be classified according to symptoms based on a sub-disease vocabulary group 430 for each symptom included in the disease vocabulary group (disease n). For example, the disease n includes symptom 1, symptom 2, and symptom 3, and the disease vocabulary group (disease n) 400 may include a sub-disease vocabulary group for symptom 1 (symptom 1), a sub-disease vocabulary group for symptom 2 (symptom 2), and a sub-disease vocabulary group for symptom 3 (symptom 3) as sub-disease vocabulary groups 430 for each symptom. The patient analysis basic sentence 420 may be classified into patient analysis basic sentence group 440 for each symptom based on the sub-disease vocabulary group 430. For example, the patient analysis basic sentence group 440 may be classified into a patient analysis basic sentence (symptom 1), a patient analysis basic sentence (symptom 2), and a patient analysis basic sentence (symptom 3). The classified patient analysis basic sentence (symptom n) may be determined as the patient analysis basic sentence group (symptom n) 440.

The patient analysis basic sentence group (symptom n) 440 may be rearranged considering time series. When there is a word whose chronological order may be determined in a sentence, the patient analysis basic sentence (symptom n) may be rearranged within the patient analysis basic sentence group (symptom n) 440 in consideration of chronological order.

A patient analysis basic information (type 2) 450 may be generated based on the patient analysis basic sentence group (symptom n) 440 and the temporally rearranged patient analysis basic sentence (symptom n). Symptom data of a current user may be extracted based on the patient analysis basic sentence (symptom n), and the user symptom data may generate the patient analysis basic information (type 2) 450. In addition, when there is a relationship between symptoms, the patient analysis basic information (type 2) 450 considering the relationship between the patient analysis basic sentences (symptom n) corresponding to different symptoms may be generated.

The patient analysis basic information (type 2) 450 generated in the above manner may be input to the voice data analysis AI engine (type 2) to generate the report basic information. The report basic information may be information in which the patient analysis basic information (type 2) 450 is classified according to detailed items to be reported.

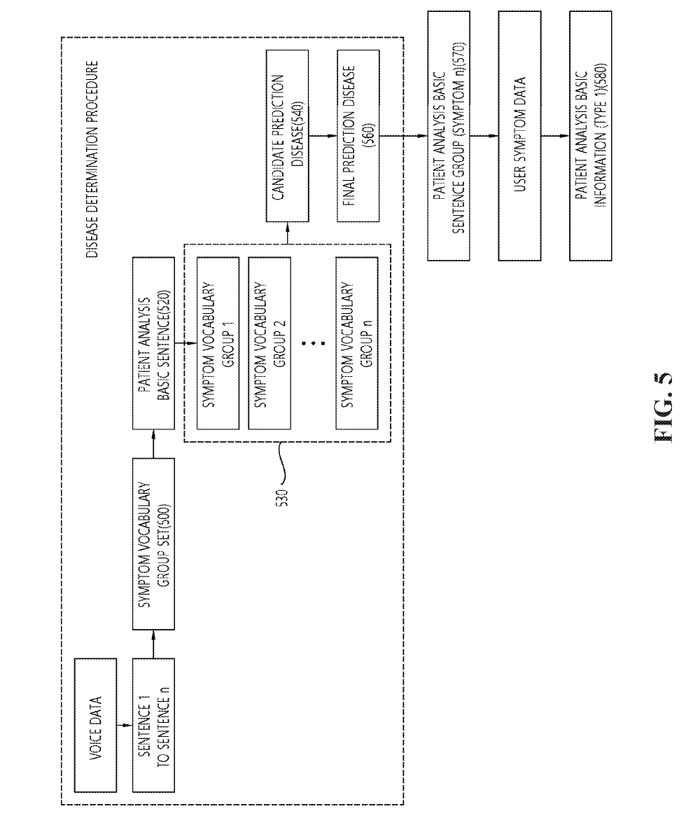

FIG. 5 is a conceptual diagram illustrating a method of generating a report according to an embodiment of the present invention.

In FIG. 5, a method of determining patient analysis basis information for report generation is disclosed. In particular, a method of generating patient analysis basic information (type 1) when there is no information on a disease already possessed by a user is disclosed.

Referring to FIG. 5, in the case of the patient analysis basic information (Type 2) described in FIG. 4, since a report is generated based on a patient's disease that has already been determined, the patient analysis basic information (type 2) may be generated without a disease determination procedure.

In order to generate patient analysis basic information (type 1) 580, a disease determination procedure of primarily determining a user's disease may be performed.

### 1. Disease Determination Procedure

The user voice data may be textualized. The textualized voice data may be divided into a plurality of sentences. In the case of the patient analysis basic information (type 1) 580, since there is no user disease data for a user disease that has already been confirmed, first, among a plurality of sentences, a sentence highly related to the symptom vocabulary group set 500 may be classified as a patient analysis basic sentence 520.

Some of the plurality of sentences may be classified based on the symptom vocabulary group set 500 as the patient analysis basic sentences 520 in consideration of symptoms. The symptom vocabulary group set 500 may include a plurality of symptom vocabulary groups 530 in which vocabularies related to symptoms are grouped. The symptom vocabulary group 530 is a group for vocabularies highly related to each symptom and may be defined for various symptoms. When the symptom is a headache, words such as headache, migraine, and sore bones may be included in the corresponding symptom vocabulary group 530. One patient analysis basic sentence 520 may correspond to different symptom vocabulary groups 530.

In the present invention, the symptom vocabulary group 530 corresponding to the patient analysis basic sentence may be determined, and a prediction of the possibility of disease may be performed based on the determined symptom vocabulary group 530. For example, when the patient analysis basic sentence 520 corresponds to symptom vocabulary group a, symptom vocabulary group b, and symptom vocabulary group c, a disease that may have the symptom vocabulary group a, the symptom vocabulary group b, and the symptom vocabulary group c may be determined as the candidate prediction disease 540.

After the candidate prediction disease 540 is determined, a final prediction disease 560 may be determined based on the patient analysis basic sentence 520 corresponding to the symptom vocabulary groups 530. The final prediction disease 560 may be determined in consideration of the relationship between the basic patient analysis sentences, a degree of relation between words included in the patient analysis basic sentence and a specific disease, and severity of a specific symptom included in the patient analysis basic sentence, and the like.

### 2. Generation of Patient Analysis Basic Information (Type 1) 580

When the final prediction disease is determined, a patient analysis basic sentence group (symptom n) 570 for the final prediction disease may be determined in the same manner as the procedure described above in FIG. 4, and the patient analysis basic information (type 1) 580 may be generated based on the sentence group (symptom n) 570.

When there is no candidate prediction disease 540, the patient analysis basic information (type 1) 580 may be generated based only on the patient analysis basic sentence 520 corresponding to a specific symptom.

The patient analysis basic information (type 1) 580 generated in the above manner may be input to the voice data analysis AI engine (type 1) to generate the report basic information. The report basic information may be information in which the patient analysis basic information (type 1) 580 is classified according to detailed items to be reported.

According to an embodiment of the present invention, when a collision occurs between patient analysis basic information, the reliability between the patient analysis basic information may be determined to process the collision. When a collision occurs, a report may be generated based on patient analysis basic information having relatively high reliability. The reliability of the patient analysis basic information may be determined in consideration of the degree of matching with the previous patient analysis basic information, the degree of overlap of the patient analysis basic information, and the like.

In addition, the patient analysis basic information equal to or less than a certain reliability may be set not to be included in a specific report in consideration of the reliability of the patient analysis basic information according to a report subject (for example, doctor, guardian, insurance company).

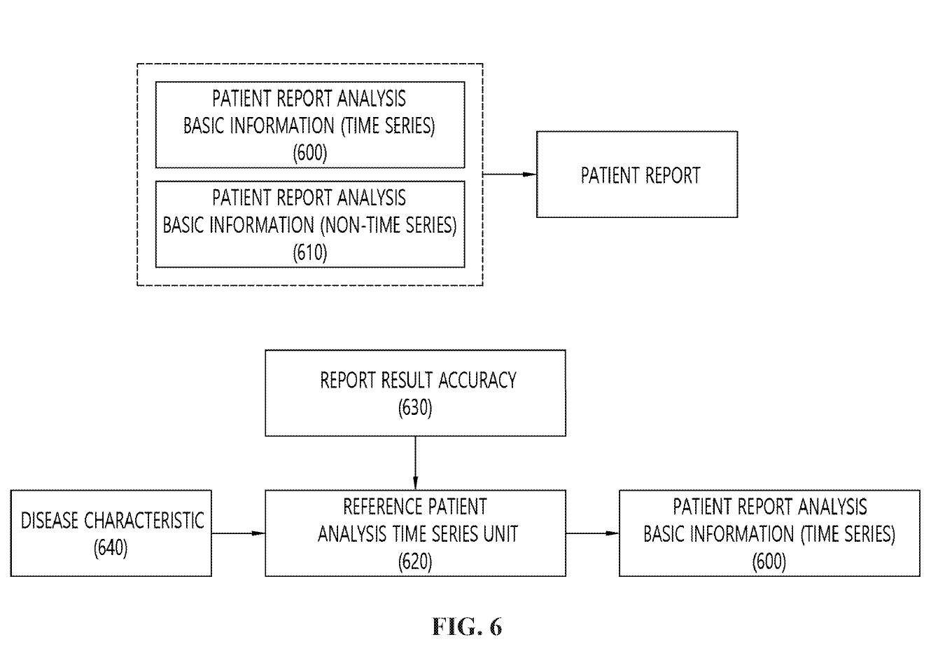

FIG. 6 is a conceptual diagram illustrating a method of generating patient report information according to an embodiment of the present invention.

FIG. 6 discloses a method of generating patient report information based on time-series information.

Referring to FIG. 6, the patient report generation engine may generate a patient report in consideration of a relationship between the existing patient analysis basic information.

The patient analysis basic information for generating a patient report may be classified into patient analysis basic information (time series) 600 that is time-series information and patient analysis basic information (non-time series) 610 that is non-time series information. The patient analysis basic information (time series) 600 may be information for generating a patient report by additionally considering previously received previous patient analysis basic information. The basic patient analysis information (non-time series) 610 may be information for generating a patient report in consideration of only the current patient analysis basic information.

In the case of the basic patient analysis information (time series) 600, it is determined whether there is the previous basic patient analysis information, and when there is the previous patient analysis basic information, the previous patient analysis basic information may be reflected when generating the current report. The time series unit of the patient analysis basic information (time series) 600 may be adaptively determined in consideration of report result accuracy and disease characteristics.

A minimum data unit for generating a report may be determined as a reference patient analysis time series unit 620 of the patient analysis basic information (time series) 600. The reference patient analysis time series unit 620 may be defined in consideration of disease characteristics. In the case of a specific disease, a time required to accumulate the patient analysis information for diagnosis or report may be defined, and based on the definition, a reference patient analysis time series unit 620 may be defined. In addition, the reference patient analysis time series unit 620 may be defined to have report result accuracy greater than or equal to a threshold value. The report result accuracy 630 may be determined in consideration of whether an item generated as a report matches an actual user's state.

The reference patient analysis time series unit 620 may be adjusted in consideration of the report result accuracy 630. In an embodiment of the present invention, the characteristics of the reference patient analysis time series unit 620 may be changed in consideration of the report result accuracy 630. For example, even when the amount of patient analysis basic information is relatively reduced due to the increase in the report result accuracy 630 due to the increase in the performance of the AI engine or the time series unit of the patient analysis basic information is reduced, if it is possible to generate report results with accuracy greater than or equal to the threshold value, the reference patient analysis time series unit 620 may be reduced. That is, the characteristics of the reference patient analysis time series unit 620 may be newly set based on the feedback on the report result.

According to one embodiment, the patient report generating device calculates the reference patient analysis time-series unit 620 as a minimum data range required to satisfy a target accuracy corresponding to the identified disease characteristic. Specifically, the processor may execute an algorithm that compares estimated report accuracy of the patient analysis basic information (time series) against a pre-stored accuracy threshold associated with the disease. If the estimated report accuracy of the patient analysis basic information (time series) is below the threshold, the processor increases the reference time-series unit to expand the range of the previous patient analysis basic information subject to analysis; conversely, if the estimated report accuracy of the patient analysis basic information (time series) meets or exceeds the threshold, the processor sets the reference time-series unit to a minimal duration. Through this calculation, the reference time-series unit is defined to ensure that the generated report meets the target accuracy while minimizing the temporal range of data required for processing.

Based on the calculated reference time-series unit, the patient report generating device dynamically allocates a portion of memory (e.g., a specific buffer size) corresponding strictly to the reference time-series unit, rather than initializing a memory block for the entire history of the user. Subsequently, the processor selectively retrieves only the patient report basic information falling within the calculated reference time-series unit from the database and loads it into the allocated memory portion. By dynamically adjusting the data load in this manner, the memory allocation is significantly reduced compared to conventional systems that indiscriminately load the entire historical dataset regardless of the required accuracy, thereby optimizing the computing resources of the device.

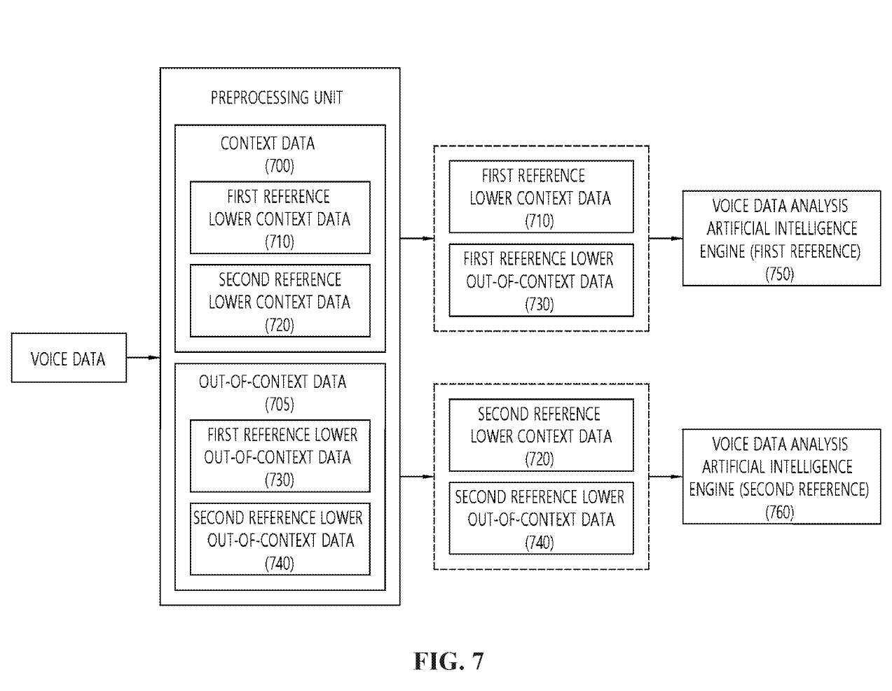

FIG. 7 is a conceptual diagram illustrating a method of generating a patient report based on user voice data according to an embodiment of the present invention.

FIG. 7 discloses a method of generating a patient report based on contextual characteristics and out-of-context characteristics in voice data input by a user.

Referring to FIG. 7, the context characteristics and the out-of-context characteristics may be classified in voice data input by a user, and based on context data 700 and out-of-context data 705, the user's disease (e.g., dementia, migraine, etc.) may be predicted and the disease may be monitored. A patient report may be generated based on disease prediction and disease monitoring results.

The preprocessing unit may extract the context data 700 and the out-of-context data 705 based on voice data. The context data may include information on characteristics generated in context, such as sentence completeness, word composition, and vocabulary. The out-of-context data may include information on out-of-context characteristics (for example, tone, pitch, a stuttering level, etc.).

Voice data analysis AI engines 750 and 760 may receive context data 700 and out-of-context data 705 and generate user symptom data of a user. The user symptom data may include disease prediction data and disease monitoring data. For example, the prediction of the possibility of a user's migraine may be performed based on the voice data analysis AI engines 750 and 760, and migraine prediction data and migraine monitoring data may be provided as user symptom data.

The context data 700 may include a plurality of lower context data, and the out-of-context data 705 may include lower out-of-context data.

In the present invention, two pieces of reference lower context data (first reference lower context data 710 and second reference lower context data 720) may exist in order to extract user's lower context data.

The first reference lower context data 710 may be lower context data commonly possessed by users having a critical ratio (e.g., 80%) or more among users having a specific disease. For example, the first reference lower context data 710 may be lower context data commonly appearing in users having dementia or migraine. The first reference lower context data 710 may be adaptively changed according to the accumulation of the user voice data. The first reference lower context data may also be set as a separate default value in consideration of a user's age, educational background, sex, and the like.

The second reference lower context data 720 may be lower context data other than the first reference lower context data 710. The second reference lower context data 720 may be lower context data observed in a specific user or a specific user group having a specific disease.

The user voice data may be analyzed based on the first reference lower context data 710 and the second reference lower context data 720, and the user symptom data may be generated based on the analysis result.

Similarly, in the present invention, two pieces of reference lower out-of-context data (first reference lower out-of-context data 730 and second reference lower out-of-context data 740) may exist in order to extract the user's lower out-of-context data.

The first reference lower out-of-context data 730 may be lower out-of-context data commonly possessed by users having a critical ratio (e.g., 80%) or more among users having a specific disease. For example, the first reference lower out-of-context data 730 may be lower out-of-context data commonly appearing in users having dementia or migraine. The first reference lower out-of-context data 730 may be adaptively changed according to the accumulation of the user voice data. The first reference lower out-of-context data 730 may also be set as a separate default value in consideration of a user's age, educational background, sex, and the like.

The second reference lower out-of-context data 740 may be lower out-of-context data other than the first reference lower out-of-context data 730. The second reference lower out-of-context data 740 may be lower out-of-context data observed in a specific user or a specific user group having a specific disease.

The user voice data may be analyzed based on the first reference lower out-of-context data 730 and the second reference lower out-of-context data 740, and the user symptom data may be generated based on the analysis result.

According to an embodiment of the present invention, the voice data analysis AI engine may be separately implemented as the voice data analysis AI engine (first reference) 750 for analyzing the first reference lower context data 710 and the first reference lower out-of-context data 730 and the voice data analysis AI engine (second reference) 760 for analyzing the second reference lower context data 720 and the second reference lower out-of-context data 740.

According to an embodiment of the present invention, in order to determine the first reference lower context data 710 and the first reference lower out-of-context data 730, user feature vectors (for example, vocabulary vector, sound feature vector, etc.) of all users having a specific disease may be located on the embedding plane. The user feature vector may be a vector extracted based on the user voice data.

The user feature vectors that users of critical criteria commonly have on the embedding plane are changed according to the accumulation of the user feature vectors of user data (or user voice data) having a disease, and thus, a vector corresponding to the first reference lower context data 710 and a vector corresponding to the data 730 other than the first reference lower context may be changed. As described above, when the first reference lower context data 710 and the first reference lower out-of-context data 730 are changed according to the accumulation of the user feature vectors of all users having a specific disease, the pool of the training data of the voice data analysis AI engine (first reference) 750 and the voice data analysis AI engine (second reference) 760 may be changed and updated.

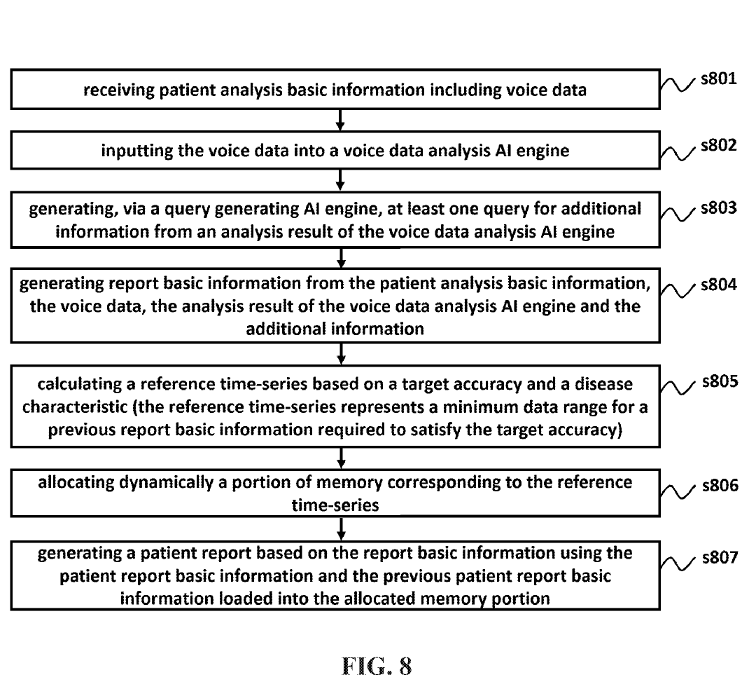

Referring now to FIG. 8, FIG. 8 is a flowchart illustrating a method for patient self-reporting based on a voice using artificial intelligence according to another embodiment of the present invention. According to an exemplary embodiment, the method may include: receiving patient analysis basic information including voice data (s801); inputting the voice data into a voice data analysis AI engine (s802); generating, via a query generating AI engine, at least one query for additional information from an analysis result of the voice data analysis AI engine (s803); generating report basic information from the patient analysis basic information, the voice data, the analysis result of the voice data analysis AI engine and the additional information (s804); calculating a reference time-series based on a target accuracy and a disease characteristic (the reference time-series represents a minimum data range for a previous report basic information required to satisfy the target accuracy) (s805); allocating dynamically a portion of memory corresponding to the reference time-series (s806); and generating a patient report based on the report basic information using the patient report basic information and the previous patient report basic information loaded into the allocated memory portion (s807).

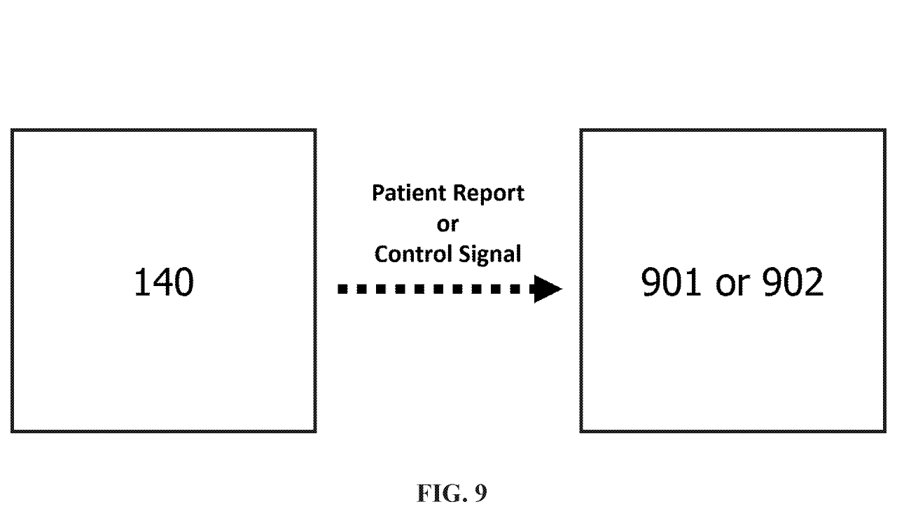

Further, referring now to FIG. 9, according to an exemplary embodiment, the patient report generated by the patient report generating device 140 or a control signal based on the generated patient report may be transmitted to a pharmaceutical mixer 901 or a supplemental device 902.

Herein, the supplemental device 902 may encompass a broad range of medical and healthcare entities capable of providing therapeutic interventions based on the generated patient report. For instance, the supplemental device 902 may include a digital therapeutic (DTx) platform configured to deliver evidence-based therapeutic interventions via software, such as mobile applications for cognitive behavioral therapy (CBT), sleep management software, or personalized lifestyle modification programs.

Furthermore, the supplemental device 902 is not limited to software but may also include hardware devices capable of automated medication delivery or patient monitoring. Examples of such hardware include automated drug delivery systems (e.g., smart insulin pumps, automated pill dispensers, smart inhalers), wearable health devices (e.g., smart belts, bio-patches), user computing devices for executing the therapeutic software (e.g., smartphones, tablets, VR/AR headsets), and environmental control systems for therapeutic purposes.

The embodiments of the present invention described above may be implemented in the form of program instructions that can be executed through various computer units and recorded on computer readable media. The computer readable media may include program instructions, data files, data structures, or combinations thereof. The program instructions recorded on the computer readable media may be specially designed and prepared for the embodiments of the present invention or may be available instructions well known to those skilled in the field of computer software. Examples of the computer readable media include magnetic media such as a hard disk, a floppy disk, and a magnetic tape, optical media such as a compact disc read only memory (CD-ROM) and a digital video disc (DVD), magneto-optical media such as a floptical disk, and a hardware device, such as a ROM, a RAM, or a flash memory, that is specially made to store and execute the program instructions. Examples of the program instruction include machine code generated by a compiler and high-level language code that can be executed in a computer using an interpreter and the like. The hardware device may be configured as at least one software module in order to perform operations of embodiments of the present invention and vice versa. Thus, for example, the prescription management device may include a processor and a memory including computer program code, where the memory and the computer program code are configured, with the processor, to cause the device to perform the functions of the steps or the method of providing a combination of medications and a user with an intake routine for the combination of medications. Also, the term “processor” is synonymous with terms like controller and computer and “should be understood to encompass not only computers having different architectures such as single/multi-processor architectures and sequential (Von Neumann)/parallel architectures but also specialized circuits such as field-programmable gate arrays (FPGA), application specific circuits (ASIC), signal processing devices and other devices.

While the present invention has been described with reference to specific details such as detailed components, specific embodiments and drawings, these are only examples to facilitate overall understanding of the present invention and the present invention is not limited thereto. It will be understood by those skilled in the art that various modifications and alterations may be made.

Therefore, the spirit and scope of the present invention are defined not by the detailed description of the present invention but by the appended claims, and encompass all modifications and equivalents that fall within the scope of the appended claims.

1. A non-transitory computer-readable storage medium having computer executable instructions stored thereon, which when executed by one or more processors, cause the one or more processors to perform operations comprising:
receiving patient analysis basic information including voice data;
inputting the voice data into a voice data analysis AI engine;
generating, via a query generating AI engine, at least one query for additional information from an analysis result of the voice data analysis AI engine;
generating report basic information from the patient analysis basic information, the voice data, the analysis result of the voice data analysis AI engine and the additional information;
calculating a reference time-series based on a target accuracy and a disease characteristic, wherein the reference time-series represents a minimum data range for a previous report basic information required to satisfy the target accuracy;
allocating dynamically a portion of memory corresponding to the reference time-series; and
generating a patient report based on the report basic information using the patient report basic information and the previous patient report basic information loaded into the allocated memory portion.

2. A patient report system for patient self-reporting based on a voice using artificial intelligence (AI), comprising:
a patient analysis device configured to:
receive patient analysis basic information;
generate, via a query generating AI engine, at least one query for additional information from an analysis result of the voice data analysis AI engine, wherein the patient analysis basic information includes voice data; and
input the voice data to a voice data analysis AI engine and generates a report basic information;
a patient report generating device configured to:
calculate a reference time-series based on a target accuracy and a disease characteristic, wherein the reference time-series represents a minimum data range for a previous report basic information required to satisfy the target accuracy;
allocate dynamically a portion of memory corresponding to the reference time-series; and
generate a patient report based on the report basic information using the patient report basic information and the previous patient report basic information loaded into the allocated memory portion.

3. The system of claim 2 further comprises an information security device, wherein the information security device is implemented to secure information generated from a user device, the patient analysis device and the patient report generating device.

4. The system of claim 2, wherein the voice data analysis AI engine comprises:
a first engine operating with only the voice data and without considering a medical history record of the user;
a second engine operating with considering both the voice data and the medical history record of the user; and
work results of both of the first engine and the second engine are utilized for the patient report.

5. The system of claim 2, wherein the query generating AI engine generates the at least one query based on:
at least one part of predetermined information being unable to be filled on the patient report based on the voice data; and
a reliability value being less than a predetermined range for an answer that generates the patient report with the voice data.

6. The system of claim 2,
wherein the voice data analysis AI engine receives a preprocessed voice data, the preprocessed voice data includes at least one tag of a predetermined disease related to sentences and words of the voice data, and
the sentences and words of the voice data are grouped based on the at least one tag for the predetermined disease.
